(19) United States
(12) Patent Application Publication
(10) Pub. No.: US 2015/0327779 A1
(43) Pub. Date: Nov. 19, 2015

(54) SYSTEM AND METHOD FOR MONITORING BLOOD FLOW CONDITION IN REGION OF INTEREST IN PATIENT'S BODY

(71) Applicant: OR-NIM MEDICAL LTD., Kfar Saba (IL)

(72) Inventors: Ilan BRESKIN, Tel-Aviv (IL); Noam RACHELI, Hadera (IL); Michal BALBERG, Jerusalem (IL)

(21) Appl. No.: 14/652,965

(22) PCT Filed: Dec. 18, 2013
(86) PCT No.: PCT/IL2013/051029

§ 371 Date: Jun. 17, 2015

Related U.S. Application Data
(60) Provisional application No. 61/738,768, filed on Dec. 18, 2012.

(51) Int. Cl. A61B 5/026; A61B 8/08; A61B 8/06; A61B 5/021; A61B 5/00

(52) U.S. Cl. CPC A61B 5/026; A61B 5/021; A61B 5/4064; A61B 8/06; A61B 5/0095; A61B 8/0808

(57) ABSTRACT

A system and method are presented for use in monitoring blood flow conditions in a region of interest in a patient's body, such as a brain or kidney region. The monitoring system comprises a blood flow sensing system, and a control unit configured for communication with the sensing system to operate and to process and analyze output data of the sensing system. The blood flow sensing system is configured and operable for measuring a blood flow parameter from a first region being a region of interest in a patient's body and generating first measured data indicative thereof, and measuring a blood flow parameter in a second region being a tissue region outside the brain region and generating second measured data indicative thereof The control unit is configured for operating the sensing system for carrying out substantially simultaneous measurements on the region of interest and the tissue region outside the region of interest and recording the first and second measured data, and configured and operable for determining a predetermined function characterizing a relation between the first measured data and the second measured data, and generating output data indicative of said relation, being indicative of the blood flow condition in the region of interest.

### TECHNOLOGICAL FIELD AND BACKGROUND

The present invention is generally in the field of medical devices, and relates to a system and method for monitoring blood flow parameters.

Monitoring cerebral blood flow to the brain is critical in situations where cerebral perfusion may be impaired. This includes situations where there is a risk of reduced perfusion for patients suffering a traumatic brain injury, a stroke or under general anesthesia.

For example, U.S. Pat. No. 8,277,385 describes a method and apparatus for assessment of hemodynamic and functional state of the brain. This technique includes non-invasive measurement of intracranial pressure, assessment of the brain's electrical activity, and measurement of cerebral blood flow, as well measuring the volume change in the intracranial vessels with a near-infrared spectroscopy or other optical method, measuring the volume change in the intracranial vessels with rheoencephalography or other electrical method, and measuring the brain's electrical activity using electroencephalography. To this end, a change in volume of blood in the jugular veins of the subject is measured; a change in volume of blood in one or more intracranial veins of the subject is measured; and a ratio of the change in volume of the one or more intracranial veins to the change in volume of the one or more jugular veins is determined, wherein changes in this ratio inversely corresponds to changes in the intracranial pressure of the subject.

### GENERAL DESCRIPTION

The present invention provides a novel technique for monitoring the condition of a region of interest, such as brain and kidney, to obtain information about adequacy of brain/kidney perfusion and impairment of the autoregulation function. This is carried out by continuously comparing between blood flow to the brain/kidney and the measures of blood flow or blood pressure on other tissue with an intact flow.

More specifically, the present invention provides a monitoring system capable of determining and displaying data indicative of a relation between several blood flow signals. The monitoring system comprises: a sensing system for sensing a first blood flow in a first region being the region of interest and sensing a second blood flow in a second region being a tissue region outside the region of interest; and a control utility which is connectable to (is in signal/data communication with) the sensing system to operate it to perform substantially simultaneous measurements on the first and second regions and record first and second measured data indicative of the first and second blood flows respectively. The control utility is preprogrammed for calculating a predetermined function characterizing a relation between the first and second measured data which is indicative of impaired or intact autoregulation in the region of interest.

Autoregulation is a mechanism that keeps blood flow (to the brain or kidney) constant while the blood pressure changes within a certain range of blood pressures. By measuring a relationship between changes in blood flow and changes in blood pressure (primarily mean arterial pressure) one can determine the state of autoregulation in particular whether autoregulation function is impaired or intact within a certain blood pressure range. If a correlation exists between the measurements, or they have a certain phase relationship, autoregulation is impaired within that blood pressure range.

The second region being a tissue region outside the region of interest is generally selected as a tissue region where a blood flow varies linearly or with a known function relative to blood pressure.

The present invention is aimed at monitoring the condition of a region of interest in brain or kidney. It should therefore be noted that any description provided herein with respect to the brain, can be applied to the kidney using the same apparatus and methods.

In some embodiments, the predetermined function characterizing the relation between the first and second measured data is a correlation function. For example, the functional relation comprises at least one of the following: a moving correlation coefficient, a phase delay, or a cross correlation between the first measured data and the second measured data.

The tissue region outside the brain from which the second data is sensed may be chosen such that blood flow in this tissue region depends linearly on the blood pressure.

In some embodiments, the sensing system includes first and second sensor units for non-invasively sensing respectively the first cerebral blood flow and the second blood flow in a tissue region outside the brain. In some other embodiments, the sensing system includes a single sensor for measuring both the non-brain and brain vasculature.

The sensing system may be configured for invasive and/or non-invasive measurements of the blood flow.

According to another broad aspect of the invention, there is provided a control unit for use in a blood flow measurement system, the control unit comprising: a data input utility for receiving first and second measured data corresponding to simultaneously measured blood flow parameter from a region of interest in a patient's body and from a body tissue region outside the region of interest; and a processor utility configured for processing the first and second measured data and determining a predetermined function characterizing a relation between the first measured data and the second measured data, and generating output data indicative of said relation, which is indicative of the blood flow condition in the region of interest.

According to yet another broad aspect of the invention, there is provided a method for use in monitoring blood flow conditions, the method comprising:

providing first and second measured data corresponding to simultaneously measured blood flow parameter from a region of interest in a patient's body and from a body tissue region outside the region of interest,

processing the first and second measured data and determining a predetermined function characterizing a relation between the first measured data and the second measured data, and generating output data indicative of said relation, which is indicative of the blood flow condition in the region of interest.

### BRIEF DESCRIPTION OF THE DRAWINGS

FIG. 1 shows schematically a monitoring system of the present invention in its operative position being placed with respective to the measurement regions on the patient body.

FIG. 2 is a block diagram illustrating the operation of the monitoring system of the invention;

FIG. 3 shows schematically the operation principles of a monitoring system of the present invention using one sensor unit;

FIG. 4 illustrates a monitoring system of the invention according to a specific not limiting example, utilizing a sensing system including laser Doppler probes;

FIG. 5 illustrates yet another example of the monitoring system of the invention where the sensing system is configured for non-invasive measurements utilizing ultrasound tagging of light; and

FIG. 6 illustrates an example for a measurement carried out with the monitoring system of the invention.

### DETAILED DESCRIPTION OF THE EMBODIMENTS

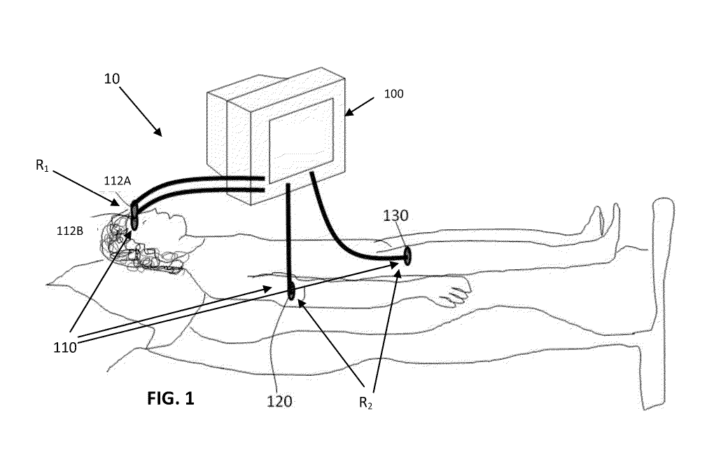

Reference is made to FIG. 1 exemplifying schematically a monitoring system 10 of the present invention. As shown, a monitoring system 10 is provided being configured and operable to collect information about blood flow in tissue. The monitoring system 10 includes a sensing system 110 configured and operable for sensing a cerebral blood flow (constituting a first blood flow in a region of interest) and a blood flow in a tissue region outside the brain (a second blood flow outside the region of interest); and a control utility 100 connectable to the sensing system 110.

The sensing system 110 includes a required number of blood flow sensor units configured for invasive and/or non-invasive blood flow measurements. In the present not limiting example of FIG. 1, four such sensor units 112A, 112B, 120, 130 are shown, while any two of them may be chosen for measurements, with one measuring from the brain and the other on another tissue region outside the brain. Also, in the present not limiting example, the connection between the control utility 100 and the sensor units is by wires, but it should be understood that the principles of the invention are not limited to this example, and any known suitable wireless connection (RF, IR, acoustic, etc.) can be used as well, in which case the sensing system 110 and the control unit 100 are equipped with appropriate communication/formatting utilities.

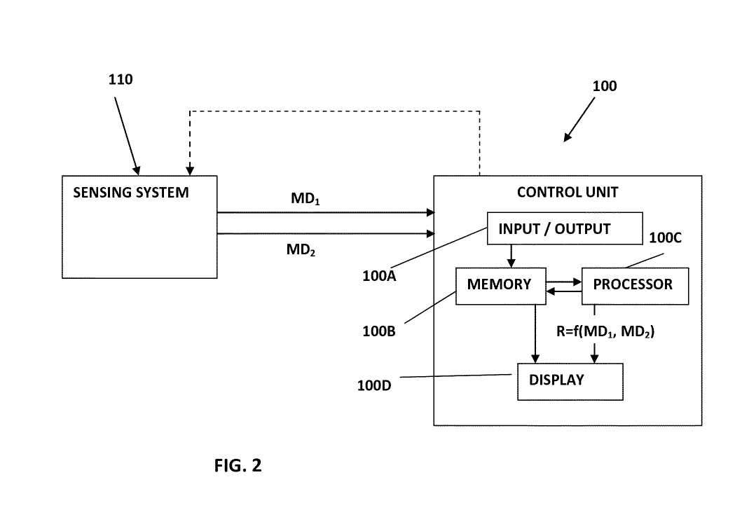

As shown in FIG. 2, by way of a block diagram, the control unit 100 is typically a computing system including inter alia such main utilities as data input/output utilities 100A, memory 100B, processor 100C, and possibly also a display 100D. Measured data from the blood flow sensing system, including the first and second measured data pieces MD₁ and MD₂, is received, and analyzed, and the results of the data analysis and possibly also the measured data itself may be displayed on graphical user interface of the display 100D.

Turning back to FIG. 1, in the present example, the sensing system 110 includes one or more blood flow sensors 112A and/or 112B applied to the head of the person (constituting a brain region R₁), such that it is operable to collect and measure data indicative of a cerebral blood flow (first measured data); and one or more other probe/sensor units 120 and/or 130 applied to another region/tissue of the body (constituting a tissue region R₂ outside the brain region R₁) for measuring blood flow in said region (second measured data). For example, sensor 120 is applied to the upper arm, and sensor 130 is applied to the lower limbs It should be understood that more than two sensors can be applied to the head (brain region) or to other regions on the body; or as will be exemplified further below a single sensor unit may be used for all the flow measurements.

The tissue volume of the region R₂ outside the brain region is preferably chosen such as to exhibit a linear relation function between the measured blood flow and the blood pressure (mean, systolic or diastolic) of the person, or a linear relation function between changes in blood pressure and changes in measured blood flow. This provides a blood pressure index.

Generally, the sensing system 110 may utilize any known suitable type of blood flow sensor(s) capable of continuously measuring the blood flow either invasively or non-invasively. A non invasive sensor unit that can be used in the system of the present invention may for example be based on the principles of ultrasound tagging of light, as described for example in U.S. Pat. No. 8,143,605 and U.S. Pat. No. 8,336,391, both assigned to the assignee of the present application and incorporated herein by reference with respect to this specific example. More specifically, such a sensing system includes an acoustic unit for irradiating a region of interest with one or more acoustic tagging beams, and an optical unit for irradiating at least a portion of the region of interest with one or more beams of electromagnetic radiation of a predetermined frequency range, and detecting an electromagnetic radiation response of the region of interest. The radiation response includes electromagnetic radiation tagged by the acoustic radiation, which is indicative of at least a blood flow parameter. In some embodiments, the sensing systems based on laser Doppler principles can be used.

As shown in FIG. 2, the control unit 100 receives the first and second measured data MD₁ and MD₂ from the sensing system (e.g. from the respective sensors), and calculates a functional relationship, R=ƒ(MD₁,MD₂), between data MD₁ measured by flow sensor(s) collecting signals from the brain (112A or 112B or both) and data MD₂ measured by flow sensor(s) collecting signals from other, non-brain regions (120 or 130 or both). For example, such functional relationship may be in the form of a moving correlation coefficient, a phase delay or a cross correlation, but is not limited to these functions. The result of the calculation can be displayed as a function or as an independent index.

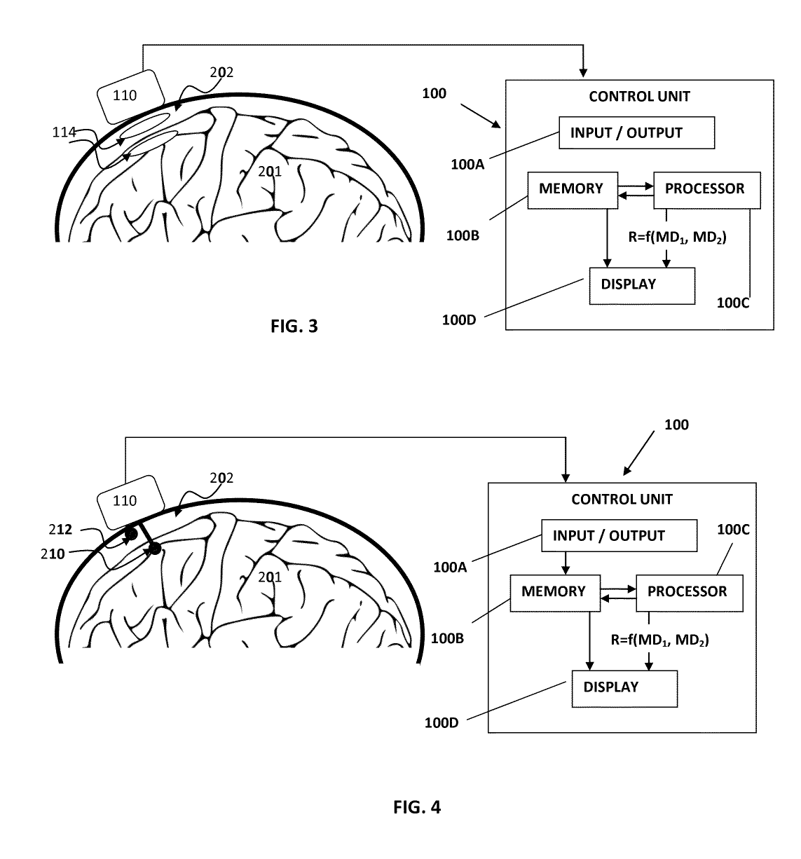

Reference is made to FIG. 3 showing an embodiment of the present invention, where the sensing system 110 includes a single blood flow sensor 114 is used to measure both non-brain and brain vasculature. The sensor 114 is configured and operable for independently applying measurements to regions of extracerebral tissue 202 and cerebral tissue 201, and the so-measured first and second data is analyzed independently. The control unit (not shown here) receives the first and second measured data from the sensing system 110, and calculates a functional relationship between data measured from the brain (region 201) and data measured from other, non-brain regions (region 202). For example, such functional relationship may be in the form of a moving correlation coefficient, a phase delay or a cross correlation, but is not limited to these functions. The result of the calculation can be displayed as a function or as an independent index. It should be noted that the sensing system 110 may include two separate sensing units that are placed on the brain, whereas one measures extracerebral tissue and the other brain tissue vasculature.

For example, FIG. 4 shows a combined sensing system 110 in more detail. Here, the sensing system includes two laser Doppler probes/sensor units 210 and 212 combined into one sensing system. Probe 210 is configured for insertion into cerebral tissue to measure cerebral blood flow variations and probe 212 measures blood flow variations in the skin. The probes 210 and 212 provide an independent measured data MD₁ and MD₂ respectively.

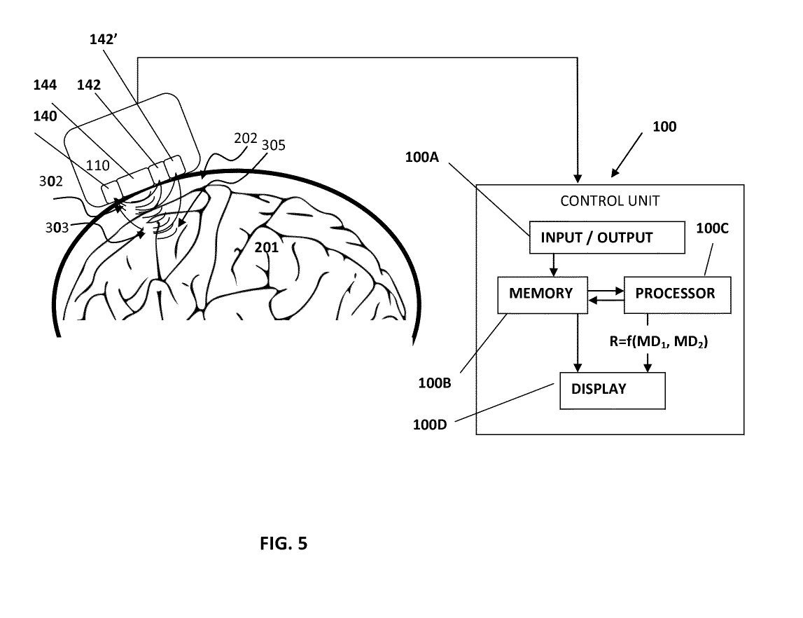

FIG. 5 shows a different configuration of the sensing system 110 that relies on non-invasive measurements utilizing ultrasound tagging of light described above. In this example, sensing system 110 comprises an illumination assembly 140, at least one detection assembly 142 and possibly additional detection assemblies (for example 142′) and an acoustic module 144. The configuration and operation of the illumination and detection assemblies and those of the acoustic module may be implemented as described in the above-indicated U.S. Pat. No. 8,143,605 assigned to the assignee of the present application, for appropriately selecting location of one or more light output ports and light input ports with respect to the acoustic port. Ultrasound waves 305 are emitted from the output port of the acoustic module. Light photons 302 emitted from illumination assembly 140 and propagate through extracerebral tissue 202 where at least a part thereof interacts with the ultrasound waves 305 and is tagged by the frequency of the acoustic radiation, and the tagged and untagged photons reach detection assembly 142. Data indicative of the output of the detection assembly is received at the control utility which is preprogrammed for analyzing the detected tagged photons and generating information about a blood flow in region 202, providing MD₂. Similarly, light photons 303 illuminate cerebral tissue 201, where they (at least a part) interact with ultrasound waves 305, and photons returned from the illuminated region reach detection assembly 142′. The control utility analyzes data indicative of tagged photons 303 and provides information about blood flow in brain region 201, providing MD₁.

It should be noted that a single detection assembly can detect photons propagating through both extracerebral and cerebral tissue, and analysis of the detected tagged signals can separate between the contribution of the two tissue regions. This can be achieved by calculating the cross correlation of the detected light signal with the generated ultrasound signal and analyzing the amplitude of this signal at different time delays from the generation of the ultrasound signal, as described in the above-mentioned U.S. Pat. No. 8,143,605.

It should be noted, although not specifically shown, that the sensing system 110 suitable for use in the present invention may utilize blood flow sensing techniques of different types, for example a combination of a laser Doppler probe and an ultrasound-tagging of light based sensing technique.

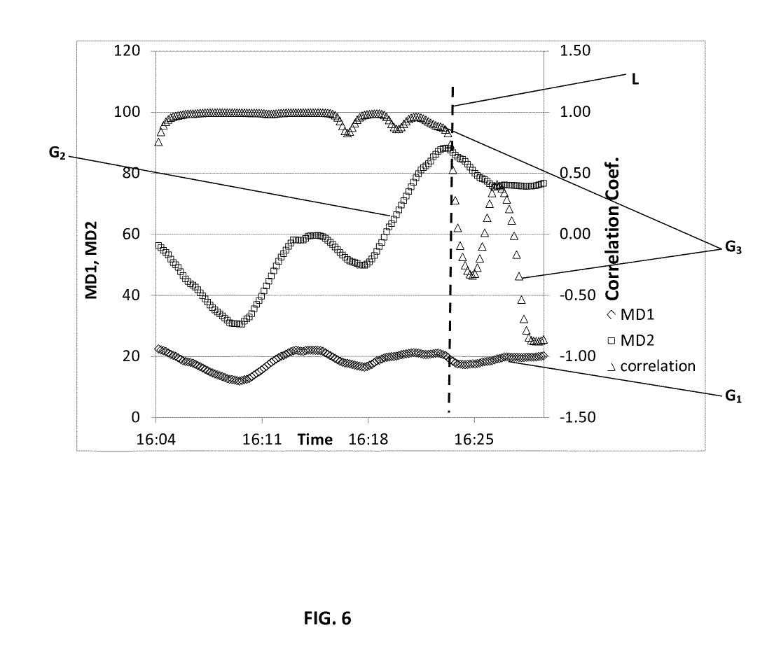

FIG. 6 shows an example for a display of a measurement with the monitoring system of the invention. Data MD₁ and MD₂ are displayed as a function of time. Graph G₁ (diamonds) represents data MD₁ collected with one sensor, graph G₂ (squares) represents data MD₂, and graph G₃ (triangles) represents a moving correlation coefficient (constituting a function ƒ of relation R between MD₁ and MD₂).

In this example, the moving correlation coefficient is calculated in the following way: each of the measured data MD₁ and MD₂ is averaged over 10 seconds interval; for every 300 seconds a correlation coefficient (r) is calculated between MD₁ and MD₂, and is displayed on the display, e.g. as a triangle; the correlation coefficient is calculated as a moving coefficient with a step of 10 seconds between each calculation. In FIG. 6, between 16:04 and 16:24 (marked with a dashed line L) the correlation coefficient is close to 1, thus indicating impaired autoregulation, whereas for the measurement period after 16:24 the correlation coefficient is lower than 1, indicating intact autoregulation for this measurement period. Data from the literature varies as to the threshold that marks the transition between intact and impaired autoregulation—a continuous display can provide continuous information as to variation of autoregulation function during treatment.

1. A system for use in monitoring blood flow conditions in a region of interest, the system comprising:
a blood flow sensing system, which is configured and operable for measuring a blood flow parameter from a first region being a region of interest in a patient's body and generating first measured data indicative thereof, and measuring a blood flow parameter in a second region being a tissue region outside the region of interest and generating second measured data indicative thereof;
a control unit configured for communication with said sensing system to operate it for carrying out substantially simultaneous measurements on the region of interest and the tissue region outside the region of interest and recording the first and second measured data, the control unit being configured and operable for determining a predetermined function characterizing a relation between the first measured data and the second measured data, and generating output data indicative of said relation, being indicative of the blood flow condition in the region of interest.

2. The system of claim 1, wherein said predetermined function is a correlation function between the first measured data and the second measured data.

3. The system of claim 1, wherein said functional relation comprises at least one of the following: a moving correlation coefficient, a phase delay, or a cross correlation between the first measured data and the second measured data.

4. The system of claim 1, wherein said predetermined function characterizing the relation between the first and second measured data is indicative of a state of autoregulation function.

5. The system of claim 1, wherein the sensing system comprises a single blood flow sensor operable by the control unit to independently perform measurements to both the region of interest and the tissue region outside the region of interest.

6. The system of claim 1, wherein the sensing system comprises at least one first blood flow sensor unit configured and operable for measuring the blood flow parameter from the region of interest and generating the first measured data indicative thereof, and at least one second blood flow sensor unit configured and operable for measuring the blood flow parameter in the tissue region outside the region of interest and generating the second measured data indicative thereof.

7. The system of claim 1, wherein the sensing system is configured for carrying out invasive or non-invasive or both types of the blood flow measurements.

8. The system of claim 1, wherein the sensing system is configured for performing the blood flow measurements on the region of interest being a region of patient's brain or kidney.

9. The system of claim 1, wherein the sensing system is configured for performing blood flow measurements on the tissue region outside the region of interest which is selected such that a blood flow in said tissue region depends substantially linearly on blood pressure.

10. A control unit for use in a blood flow measurement system, the control unit comprising:
a data input utility for receiving first and second measured data corresponding to simultaneously measured blood flow parameter from a first region being a region of interest in a patient's body and from a second region being a body tissue region outside the region of interest; and
a processor utility configured for processing the first and second measured data and determining a predetermined function characterizing a relation between the first measured data and the second measured data, said relation comprising at least one of the following: a moving correlation coefficient, a phase delay, and a cross correlation between the first measured data and the second measured data, and generating output data indicative of said relation, which is indicative of the blood flow condition in the region of interest.

11. A method for use in monitoring blood flow conditions, the method comprising:
providing first and second measured data corresponding to simultaneously measured blood flow parameter from a region of interest in a patient's body and from a body tissue region outside the region of interest,
processing the first and second measured data and determining a predetermined function characterizing a relation between the first measured data and the second measured data, and generating output data indicative of said relation, which is indicative of the blood flow condition in the region of interest.

12. The method of claim 11, wherein the tissue region outside the region of interest is selected such that a blood flow in said tissue region depends substantially linearly on blood pressure.

13. The method of claim 11, wherein said predetermined function is a correlation function between the first measured data and the second measured data.

14. The method of claim 11, wherein said functional relation comprises at least one of the following: a moving correlation coefficient, a phase delay, or a cross correlation between the first measured data and the second measured data.

15. The method of claim 11 wherein the region of interest is a brain region or a kidney region.
